(19) United States
(12) Patent Application Publication
(10) Pub. No.: US 2023/0390268 A1
(43) Pub. Date: Dec. 7, 2023

(54) METHOD FOR TREATING VIRAL INFECTIONS USING ASK1 INHIBITORS

(71) Applicant: McMaster University, Hamilton (CA)

(72) Inventors: Karen Mossman, Ancaster (CA); Wael Demian, London (CA)

(21) Appl. No.: 18/202,467

(22) Filed: May 26, 2023

Related U.S. Application Data
(60) Provisional application No. 63/348,164, filed on Jun. 2, 2022.

(51) Int. Cl. A61K 31/4439; A61P 31/18; A61P 31/14

(52) U.S. Cl. CPC A61K 31/4439; A61P 31/18; A61P 31/14

(57) ABSTRACT

The present disclosure is directed to a method for treating or preventing viral infections using ASK1 inhibitors. The present disclosure is also directed to the treatment or prevention of the symptoms, complications and/or disorders related to a cytokine storm using ASK1 inhibitors.

### RELATED APPLICATION

This disclosure claims benefit of U.S. Provisional Patent Application Ser. No. 63/348,164 filed Jun. 2, 2022, the entire contents of Provisional Application No. 63/348,164 incorporated herein by reference in its entirety.

### FIELD

The present disclosure is directed to a method for treating or preventing viral infections using apoptosis signal-regulating kinase 1 (ASK1) inhibitors. The present disclosure is also directed to the treatment or prevention of symptoms, disorders or complications related to a cytokine storm using ASK1 inhibitors.

### INTRODUCTION

Viruses induce a stress response in host cells by generating high levels of particular stress proteins or their mRNAs (Jindal and Malkovsky, 1994, Wan, Song, Li and He, 2020). Multiple stress-activated signalling pathways are upregulated that function as critical defense systems in host cells against the viral infection (Rozelle, Filone, Kedersha, and Connor, 2014, Wu, Zhang, Li, and Li, 2022). Stress-activated mitogen-activated protein kinase (MAPK) is one of various stress-activated signalling kinases that converge on c-Jun N-terminal kinases (JNK) and p38 MAP kinases (p38MAPK) that have been characterised as regulators of cellular functions, including apoptosis and inflammation, in response to a wide variety of extracellular or intracellular stress (Kyriakis and Avruch, 2001). Alternatively, the upstream regulator apoptosis signal-regulating kinase 1 (ASK1/MAP3K5) is a key mediator of reactive oxygen species (ROS)-induced JNK and p38MAPK activation under pathological conditions. ASK1 is a MAP kinase that is activated in response to proinflammatory stimuli, ROS, and other cellular stresses. ASK1 activates the MAP2K4/7 (MKK4/7)-JNK pathway and MAP2K3/6 (MKK3/6)-p38 pathway (Ichijo et al., 1997). The canonical activation of p38 MAPK occurs via dual phosphorylation of Tyr and Thr residues in a conserved TGY motif. Phosphorylation of p38 is catalyzed by the dual specificity kinases MKK3 and MKK6, which are in turn activated upon phosphorylation of Ser/Thr residues by a MAPK kinase (MAP3K) such as ASK1 (Cuenda and Rousseau 2007). Interestingly, MKK3 and MKK6 are highly selective for p38-MAPK and do not activate other MAPKs (Remy et al., 2010, Cuenda and Rousseau 2007). Moreover, MKK4 has also been shown to contribute in activation of p38 (Remy et al., 2010, Cuenda and Rousseau 2007).

### SUMMARY

The present disclosure is directed to the treatment or prevention of a viral infection in a subject in need thereof, comprising administering to the subject an effective amount of an apoptosis signal-regulating kinase 1 (ASK1) inhibitor.

In another embodiment, the present disclosure is directed to the treatment or prevention of symptoms, complications and/or disorders related to or associated with a cytokine storm in a subject in need thereof, comprising administering to the subject an effective amount of an ASK1 inhibitor.

In one embodiment, the ASK1 inhibitor is ASK1 inhibitor 10, Selonsertib, GS444217, BPyO-34, GS-459679, GS-627, K811, K812, MSC2032964A, SRT-015, EP-027315, EP-026856, or Analog 21.

In another embodiment, the viral infection is a severe acute respiratory syndrome coronavirus 2 (SARS-CoV-2) infection, a vesicular stomatitis virus (VSV) infection, a herpes simplex virus (HSV) infection, a human immunodeficiency virus infection (HIV) or Vaccinia virus (VacV) infection.

In another embodiment, the cytokine storm involves the sudden release of an excessive amount of proinflammatory cytokines resulting in elevated levels of circulating cytokines. In one embodiment, the cytokine storm can occur as a result of viral infections, therapies, other pathogens, cancers and autoimmune disorders.

Other features and advantages of the present application will become apparent from the following detailed description. It should be understood, however, that the detailed description and the specific examples while indicating preferred embodiments of the application are given by way of illustration only, since various changes and modifications within the spirit and scope of the application will become apparent to those skilled in the art from this detailed description.

### BRIEF DESCRIPTION OF THE DRAWINGS

The present disclosure will now be described in greater detail with reference to the drawings in which:

FIG. 1 shows the inhibition of ASK1 kinase decreases SARS-CoV-2 replication in one embodiment of the disclosure.

FIG. 2 shows ASK1 blockade reduces SARS-CoV-2 replication and the levels of proinflammatory cytokines, IL6, TNF-α and IL8 in one embodiment of the disclosure.

FIG. 3 shows ASK1 inhibition (by Selonsertib and GS-444217) abrogates replication of several viruses in one embodiment of the disclosure.

FIG. 4 shows ASK1 blockade inhibits HIV replication. in one embodiment of the disclosure

FIG. 5 shows ASK1 drugs have a long-term effect that diminishes VSV replication and the downstream cascade signaling of p38MAPK in one embodiment of the disclosure.

FIG. 6 shows ASK1 Drugs (Selonsertib and GS-444217) do not alter the cell viability in one embodiment of the disclosure.

FIG. 7 shows that SARS-CoV-2 and VSV replication is weakened in ASK1 depleted cells in one embodiment of the disclosure.

FIG. 8 shows the effect of ASK1 inhibition on viral replication is independent on JNK, the downstream effector of ASK1. (A) and (B) JNK inhibition does not alter VSV replication. (A) Immunoblot shows that JNK phosphorylation is reduced upon treated infected cells with Selonsertib or JNK inhibitor (SP600125). Typhon image is depicted on the top of the immunoblot. (B) Typhon image shows the VSV-GFP replication in cells treated with DMSO, Selonsertib or SP600125. Quantification of GFP signals is depicted below the scanned image. (A) and (B) Calu3 cells were infected with VSV-GFP for an hour at (A) MOI of 1 or (B) MOI of 1, 0.1 or 0.01, followed by immediately adding Selonsertib or JNK inhibitor after infection, and the cells were incubated for 24 h prior their scanning by Typhoon or lysis for immunoblotting. In the bar graph, DMSO (first bar on left), Selonsertib (middle bar), SP600125 (right bar).

FIG. 9 shows Selonsertib did not drastically affect autophagy and mTORC1 activation in VSV infected cells. (A) THF and (B) Calu3 cells were infected with mock or VSV-GFP for an hour. Then, the cells were treated with different drugs for 24 h hpi; DMSO (control), Torin1 (mTORC1 inhibitor), Selonsertib (ASK1 inhibitor, BIRB (p38MAPK inhibitor), MG132 (proteosomae suppressor) and chloroquine (CHQ, autophagy suppressor).

FIG. 10 shows Selonsertib enhances SARS-CoV-2 replication in hamster. (A) Immunoblot shows an increase in the expression levels of SARS-CoV-2 nucleoprotein. CHL-11, lung hamster cells were infected with SARS-CoV-2 virus for 1 hr followed by DMSO or Selonsertib treatment. Cells were incubated for 24 hr prior cell lysis. (B), (C) and (D) data of in vivo trial of Syrian Hamsters. (B) Percentage of weigh loss was measured for 7 days (2 days prior infection and 5 days after infection) (C) Images of IHC tissues that were stained with SARS-CoV2-N (nucleocapsid) antibody (Scale bar=100 μm). (D) Bar graph represents quantification of the IHC images. The quantification represents positive area (μm²) of SARS-CoV-2 nucleoprotein stain that were distributed in tissues collected from 6 hamsters/group.

FIG. 11 shows Selonsertib abrogates VSV and HSV replication in hamster cell line. (A) and (B) CHL-11 cells were infected either with VSV-GFP or HSV-GFP at MOI of 1, 0.1 or 0.01 followed by adding Selonsertib immediately after infection and cells were incubated for 24 h. Selonserib was added at different doses of a concentration of 25 or 50 μM. FI of GFP signals was measured and quantified by Typhoon Imager, and the signal quantification is depicted beneath the scanned images. In bar graphs, DMSO (first bar on left), Selonsertib 50 μM (middle bar), Selonsertib 25 μM (right bar).

### DESCRIPTION OF VARIOUS EMBODIMENTS

The present disclosure is directed to the treatment or prevention of a viral infection in a subject in need thereof, comprising administering to the subject an effective amount of an ASK1 inhibitor.

In another embodiment, the present disclosure is directed to the treatment or prevention of the symptoms, complications and/or disorders related to or associated with a cytokine storm in a subject in need thereof, comprising administering to the subject an effective amount of an ASK1 inhibitor.

In one embodiment, the ASK1 inhibitor is any agent, compound, drug, biological molecule (protein, polypeptide, or antibody) or medicine, that is capable of inactivating an ASK1 protein kinase.

In one embodiment, the ASK1 inhibitor is ASK1 inhibitor 10

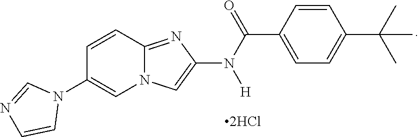 [SMILES: CC(C)(C)c1ccc(C(=O)Nc2cn3cc(-n4ccnc4)ccc3n2)cc1]

In one embodiment, the ASK1 inhibitor is GS444217

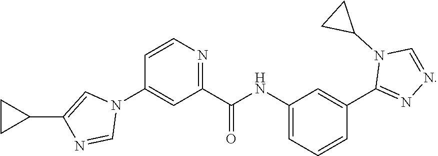 [SMILES: O=C(Nc1cccc(-c2nncn2C2CC2)c1)c1cc(-n2cnc(C3CC3)c2)ccn1]

In one embodiment, the ASK1 inhibitor is Selonsertib

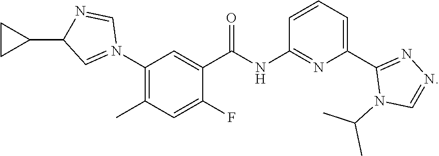

In one embodiment, the ASK1 inhibitor is BPyO-34

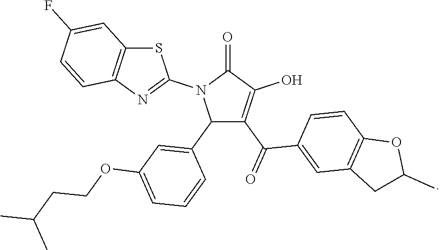 [SMILES: CC(C)CCOc1cccc(C2C(C(=O)c3ccc4c(c3)CC(C)O4)=C(O)C(=O)N2c2nc3ccc(F)cc3s2)c1]

In one embodiment, the ASK1 inhibitor is K811

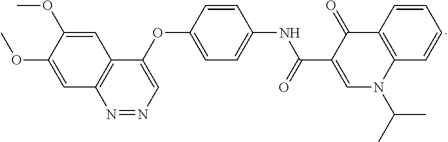 [SMILES: COc1cc2nncc(Oc3ccc(NC(=O)c4cn(C(C)C)c5ccccc5c4=O)cc3)c2cc1OC]

In one embodiment, the ASK1 inhibitor is K812

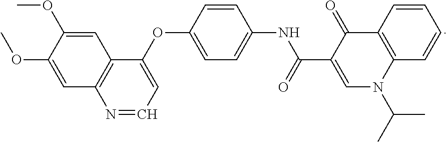 [SMILES: COc1cc2nccc(Oc3ccc(NC(=O)c4cn(C(C)C)c5ccccc5c4=O)cc3)c2cc1OC]

In one embodiment, the ASK1 inhibitor is GS-459679 (Gilead Sciences®).

In one embodiment, the ASK1 inhibitor is GS-627 (Gilead Sciences®)

In one embodiment, the ASK1 inhibitor is MSC2032964A

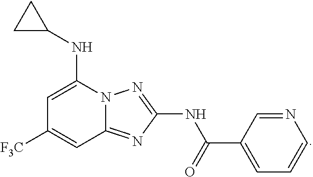 [SMILES: O=C(Nc1nc2cc(C(F)(F)F)cc(NC3CC3)n2n1)c1cccnc1]

In one embodiment, the ASK1 inhibitor is SRT-015 (Seal Rock Therapeutics®).

In one embodiment, the ASK1 inhibitor is TC ASK 10

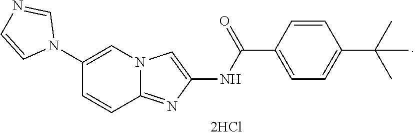 [SMILES: CC(C)(C)c1ccc(C(=O)Nc2cn3cc(-n4ccnc4)ccc3n2)cc1]

In one embodiment, the ASK1 inhibitor is EP-027315 (Entana Pharma®).

In one embodiment, the ASK1 inhibitor is EP-026856 (Entana Pharma®).

In one embodiment, the ASK1 inhibitor is Analog 21

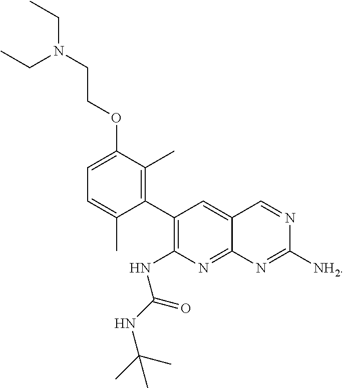 [SMILES: CCN(CC)CCOc1ccc(C)c(-c2cc3cnc(N)nc3nc2NC(=O)NC(C)(C)C)c1C]

In one embodiment, the viral infection is an RNA virus infection or a DNA virus infection.

In another embodiment, the viral infection is a SARS-CoV-2 infection, a vesicular stomatitis virus infection (VSV), a herpes simplex virus infection, a human immunodeficiency virus infection or a vaccinia virus infection.

In another embodiment, the cytokine storm involves the sudden release of an excessive amount of proinflammatory cytokines resulting in elevated levels of circulating cytokines. In one embodiment, the cytokine storm can occur as a result of viral infections, therapies, other pathogens, cancers, smoking and autoimmune disorders. In another embodiment, the cytokine storm results in symptoms including fever, chills, tiredness, nausea and vomiting, diarrhea, headaches, cough, low blood pressure, joint pain, muscle pain, skin rash, shortness of breath, confusion, dizziness and difficulty swallowing. In one embodiment, treatment of the cytokine storm with an ASK1 inhibitor reduces, lessens or ameliorates any of these symptoms.

In another embodiment of the disclosure, there is included pharmaceutical compositions for the treatment or prevention of a viral infection in a subject in need thereof, comprising administering to the subject an effective amount of a pharmaceutical composition comprising an apoptosis signal-regulating kinase 1 (ASK1) inhibitor and a pharmaceutically acceptable excipient.

In further embodiments, there is included pharmaceutical compositions for the treatment or prevention of the symptoms, complications and/or disorders related to a cytokine storm in a subject in need thereof, comprising administering to the subject an effective amount of a pharmaceutical composition comprising an apoptosis signal-regulating kinase 1 (ASK1) inhibitor and a pharmaceutically acceptable excipient.

In one embodiment, a subject may be a mammal, for example, human, horse, cattle (bovine), pig, sheep, goat, dog, and other domestic animals. Preferably, the subject is a human. More preferably, the subject is a human having suffered from or who is predisposed to a viral infection or a cytokine storm.

In one embodiment, the effective amount of the ASK1 inhibitor refers to an amount of inhibitor or pharmaceutical composition comprising the ASK1 inhibitor required to achieve the goal (e.g., treating or preventing a viral infection or cytokine storm in a subject in need thereof). The effective amount of the pharmaceutical composition comprising an ASK1 inhibitor may vary depending upon the stated goals, the physical characteristics of the subject, the nature and severity of the disease or condition or symptom (of the viral infection or cytokine storm), the existence of related or unrelated medical conditions, the nature of the ASK1 inhibitor, the composition comprising the ASK1 inhibitor, the means of administering the composition to the subject, and the administration route. The pharmaceutical composition may be administered to the subject in one or multiple doses. Each dose may comprise an ASK1 inhibitor at about 0.01-5000 mg/kg, preferably about 0.1-1000 mg/kg, more preferably about 1-500 mg/kg.

In one embodiment, the pharmaceutically acceptable excipient is any compound or ingredient that is compatible with the ASK1 inhibitor and the other ingredients in a pharmaceutical formulation. Suitable excipients are known to those of skill in the art and examples are described, for example, in the Handbook of Pharmaceutical Excipients (Kibbe (ed.), 3rd Edition (2000), American Pharmaceutical Association, Washington, D.C.), and Remington's Pharmaceutical Sciences (Gennaro (ed.), 20th edition (2000), Mack Publishing, Inc., Easton, Pa.). Examples of excipients include but are not limited to fillers, extenders, diluents, wetting agents, solvents, emulsifiers, preservatives, absorption enhancers, sustained-release matrices, starches, sugars, microcrystalline cellulose, granulating agents, lubricants, binders, disintegrating agents, coloring agents, release agents, coating agents, sweetening agents, flavoring agents, perfuming agents, antioxidants, plasticizers, gelling agents, thickeners, hardeners, setting agents, suspending agents, surfactants, humectants, carriers, stabilizers, and combinations thereof.

Although the disclosure has been described in conjunction with specific embodiments thereof, if is evident that many alternatives, modifications and variations will be apparent to those skilled in the art. Accordingly, it is intended to embrace all such alternatives, modifications and variations that fall within the spirit and broad scope of the appended claims. In addition, citation or identification of any reference in this application shall not be construed as an admission that such reference is available as prior art to the present disclosure.

### EXAMPLES

The operation of the disclosure is illustrated by the following representative examples. As is apparent to those skilled in the art, many of the details of the examples may be changed while still practicing the disclosure described herein.

### Materials and Methods

KEY RESOURCES TABLE

| | | |
|---|---|---|
| REAGENT or RESOURCE | SOURCE | IDENTIFIER |

Antibodies

| | | |
|---|---|---|
| Mouse anti-β-actin | Sigma-Aldrich | Cat# A2228; |
| | | RRID: AB_47669 |
| Mouse anti-phospho p38 MAPK | BD Bioscience | Cat# 612280; |
| (T180/Y182) | | RRID: AB_399597 |
| Rabbit anti-p38 MAPK | Cell Signaling | Cat# 9212; |
| | | RRID: AB_330713 |
| Rabbit anti-GAPDH | Cell Signaling | Cat# 2118 |
| | | RRID: AB_561053 |
| Rabbit anti-phospho-MKK3 | Cell Signaling | Cat#9231 |
| (S189)/MKK6 (S207) | | RRID: AB_2140799 |
| Rabbit anti-phospho- | Cell Signaling | Cat#9156 |
| SEK1/MKK4 (S257/T261) | | RRID: AB_2297420 |
| Rabbit anti-ASK1 | Abcam | Cat# ab45178 |
| | | RRID: AB_722915 |
| Rabbit anti-phospho-ASK1 | Cell Signaling | Cat# 3765 |
| (Thr845) | | RRID: AB_2139929 |
| Mouse anti-SARS/SARS- | ThermoFisher | Cat# MA5-29981; |
| CoV-2 N | Scientific | RRID: AB_2785780 |
| Rabbit LC3B | Cell Signaling | Cat#2775; |
| | | RRID: AB_915950 |
| Rabbit anti-phospho S6K | Cell Signaling | Cat# 9234; |
| (T389) | | RRID: AB_2269803 |
| Mouse anti-S6K (p70) | Santa Cruz | Cat# sc-57324; |
| | | RRID: AB_784102 |
| Rabbit anti-rabbit 800 | LI-COR Biosciences | Cat# 926-32213; |
| | | RRID: 621848 |
| Goat anti-mouse 800 | LI-COR Biosciences | Cat# 926-32210 |
| | | RRID: AB_621842 |
| Goat anti-mouse 680 | LI-COR Biosciences | Cat# 925-68070; |
| | | RRID: AB_265112 |
| Goat anti-rabbit 680 | LI-COR Biosciences | Cat# 92568071; |
| | | RRID AB_2721181 |

Virus Strains/Cell lines

| | | |
|---|---|---|
| SARS-CoV-2/SB3 | Laboratory of Samira | (Banerjee et al., |
| | Mubareka | 2020) |
| VSV-GFP | Laboratory of Brian Lichty | (Leveille et al., 2011) |
| HSV-KOS-GFP | Laboratory of Karen | (Minaker et al., |
| | Mossman | 2005) |
| Vaccinia-EYFP | A gift of John Bell and was | (Rintoul et al., 2011) |
| | generated using | |
| | conventional recombination | |
| | methods to bifurcate the | |
| | thymidine kinase gene and | |
| | introduce EYFP fused to the | |
| | GPT selection gene in the | |
| | genome of a western reserve | |
| | strain vaccinia | |
| Virus 1 IIIB (HIV-1 IIIB)- | The H9 cell line chronically | (Popovic et al., |
| Infected H9 Cells (T-cells) | infected with HIV-1 IIIB was | 1984) |
| | obtained through the NIH | |
| | HIV Reagent Program, | |
| | Division of AIDS, NIAID, | |
| | NIH: Human | |
| | Immunodeficiency Virus 1 | |
| | IIIB (HIV-1 IIIB)-Infected H9 | |
| | Cells, ARP-400, contributed | |
| | by Dr. Robert Gallo. | |
| TZMbl cell line | The cell line was obtained | (Derdeyn et al., |
| | through the NIH HIV | 2000) |
| | Reagent Program, Division | |
| | of AIDS, NIAID, NIH: TZM-bl | |
| | Cells, ARP-8129, | |
| | contributed by Dr. John C. | |
| | Kappes, Dr. Xiaoyun Wu and | |
| | Tranzyme Inc. | |
| Calu-3 cells | ATCC: The Global | HTB-55 |
| | Bioresource Center | |
| THF cells | Laboratory of Victor | (DeFilippis et al., |
| | DeFilippis | 2010) |
| HAP1 cells (WT) and | PerkinElmer | Cat#HZGHC000093 |
| (MAP3K5 KO) | | c001 |
| CHL/IU [CHL-11] cells | ATCC: The Global Bioresource Center | CRL-1935 |

Chemicals, Peptides, and Recombinant Proteins

| | | |
|---|---|---|
| Lipofectamine | Invitrogen | Cat#L3000015 |
| Poly(I:C) | Invitrogen | Cat#tlrl-pic |
| GS-444217 | MedKoo Biosciences | Cat#564706 |
| Selonsertib (GS-4997) | MedKoo Biosciences | Cat#206450 |
| Doramapimod (Synonyms: | MedChem Express | Cat#285983-48-4 |
| BIRB 796) | | |
| SP600125 | Selleck Chemicals | Cat#S1460 |
| Torin1 | Tocris | Cat#4247 |
| MG132 | Sigma | Cat#133407-82-6 |
| Chloroquine | Sigma | Cat# 50-63-5 |
| MTT Assay Kit (Cell Proliferation) | Abcam | Cat# ab211091 |

Software and Algorithms

| | | |
|---|---|---|
| Prism software | GraphPad | https://www.graphpad.com |
| Image Studio | LI-COR Biosciences | https://www.licor.com/bio/image-studio/ |
| Adobe Illustrator | Adobe | https://www.adobe.com/products/illustrator. |
| | | html?promoid=PGRQQLFS&mv=other |
| BioRender | BioRender | https://biorender.com |
| EVOS XL Inverted Imaging Digital | Life Technologies | NA |
| Microscope | (ThermoFisher) | |
| Typhoon Imager | GE Healthcare Life Sciences | NA |

Critical Commercial Assays

| | | |
|---|---|---|
| Ssofast EvaGreen supermix | Bio-Rad | Cat#1725201 |
| iScript gDNA Clear cDNA synthesis kit | Bio-Rad | Cat#172-5035 |
| RNeasy Mini Kit | Qiagen | Cat#74106 |

Oligonucleotides

| | | |
|---|---|---|
| PrimePCR ™ SYBR ® Green Assay: | Biorad | Cat# |
| IL6, Human | | qHsaCID0020314 |
| PrimePCR ™ PreAmp for SYBR ® | Biorad | Cat# |
| Green Assay: IL8, Human | | qHsaCED004663 |
| PrimePCR ™ PreAmp for SYBR ® | Biorad | Cat# |
| Green Assay: TNF, Human | | qHsaCEP0040184 |
| Human GAPDH-F | IDT | N/A |
| (GTCTCCTCTGACTTCAACAGCG) | Corp. | |
| Human GAPDH-R | IDT | N/A |
| (ACCACCCTGTTGCTGTAGCCAA) | Corp. | |
| SARS2 UpE-F | IDT | N/A |
| (ATTGTTGATGAGCCTGAAG) | Corp. | |
| SARS2 UpE-R | IDT | N/A |
| (TTCGTACTCATCAGCTTG) | Corp. | |

### Experimental Model and Subject Details

### Calu3 and THF Cells and Viruses

Calu-3 cells (human male lung adenocarcinoma derived; ATCC) were maintained and cultured as previously mentioned (Aguiar et al., 2019). THF cells (human telomerase life-extended cells; from Dr. Victor DeFilippis' lab, (Bresnahan et al., 2000; DeFilippis et al., 2010)) were maintained in Dulbecco's modified Eagle's media (DMEM) supplemented with 10% fetal bovine serum (FBS; Sigma-Aldrich), 1× L-Glutamine, and Penicillin/Streptomycin (Pen/Strep; VWR)

HAP1 cells (WT) and (MAP3K5 KO) (PerkinElmer) were maintained in Iscove's Modified Dulbecco's Medium (IMDM) (Fisher Scientific, Cat #12-440-053) supplemented with 10% fetal bovine serum (FBS; Sigma-Aldrich), 1× L-Glutamine, and Penicillin/Streptomycin (Pen/Strep; VWR). CHL/IU [CHL-11] cell line (isolated from the lung of a female Chinese hamster) were maintained and cultured in Eagle's Minimum Essential Medium (EMEM) (ATCC CRL-1935) with 10% fetal bovine serum (FBS; Sigma-Aldrich), 1× L-Glutamine, and Penicillin/Streptomycin (Pen/Strep; VWR).

Stocks of genetically engineered vesicular stomatitis virus (VSV-GFP) carrying a green fluorescent protein (GFP) cassette (Leveille et al., 2011; Noyce et al., 2011) were stored at −80° C. HSV-GFP stocks were generated and maintained as mentioned previously (Minaker et al., 2005). Clinical isolate of SARS-CoV-2 (SARS-CoV-2/SB3) was propagated on Vero E6 cells and validated by next-generation sequencing (Banerjee et al., 2020).

Virus stocks were thawed once and used for an experiment. A fresh vial was used for each experiment to avoid repeated freeze-thaws. VSV-GFP, HSV-GFP, VacV-EYFP virus infections were performed at a multiplicity of infection (MOI) of 1, 0.1 or 0.01. SARS-CoV-2 infections were performed at MOIs of 1.

### TZMbl HIV-1 Inhibition Assay

TZMbl cell line was used for primary infection assays (Kimpton and Emerman 1992). The TZMbl cell line was maintained in DMEM supplemented with 10% fetal bovine serum (FBS) and Penicillin/Streptomycin. TZMbl cell line were grown in a humidified incubator at 37° C. in the presence of 5% CO₂. For Assay TZM-bl cells were seeded in 24 well plates and the infection were conducted in two different ways primary HIV-1 infection assay and prevention of HIV-1 replication after 24 hours.

In primary infection assays, the cells were infected for 2 hours in serum free medium with HIV-1 NL4-3 GFP with multiplicity of infection (MOI) of 1 (corresponding to 300 ng/mL of HIV-1 p24) or mock. After 2 hours of incubation, cells were either overlayed with medium containing DMSO or medium containing two different concentrations (50 μM and 25 μM) of ASK1 inhibitors (Selonsertib or GS444217). After 48 hr post infection (hpi), the HIV-1 infected fluorescent cells were imaged by Typhoon Imager (GE Healthcare Life Sciences). In prevention of HIV-1 replication assays, TZMbl cells were infected with HIV-1 NL4-3 GFP with multiplicity of infection (MOI) of 1 (corresponding to 300 ng/mL of HIV-1 p24) or mock. After 2 hours of incubation, cells were overlayed with serum containing medium. After 24 hours of incubation, medium was replaced with drug containing medium. TZMbl cells were imaged with Typhoon Imager (GE Healthcare Life Sciences) or EVOS after 48 hours of infection.

### T-Cell HIV-1 Inhibition Assay

H9 (T-cell) cell line chronically infected with HIV-1 III_{B} was grown in RPMI with 10% Fetal bovine serum. One million cells per well were added either with medium or medium containing Drug 1 and 2 individually in two different concentrations and incubated for 24 hours. After 24 hours of incubation supernatant were collected and HIV-1 titration was performed using TZMbl cells as described previously (Kimpton and Emerman 1992). Briefly, viral supernatant collected from H9 cells were serially diluted in serum free medium and added to TZMbl cells, after 2 hours of incubation TZMbl cells were overlayed with serum containing medium and further incubated for 48 hours. After 48 hours of infection, media were aspirated, and the cells were fixed and stained for β-galactosidase activity. Blue infected cells showing positive β-galactosidase activity were counted and estimated accounting for dilution.

### Immunoblotting

Calu3, THF, HAP1 or CHL-11 cells were infected with VSV, HSV or VacV at MOI of 0.1 for an hour or transfected with 100 nM of poly(I:C) (invitrogen). For drug treatment, the cells were treated with 10, 25 or 50 μM of ASK1 inhibitors (Selonsertib or GS444217) or 10 μM of p38MAPK inhibitor (BIRB) as indicated in the figure legends. Then, they lysed in a lysis buffer of (50 mM Hepes, pH 7.5, 150 mM NaCl, 1% Triton X-100, 10% glycerol, 1.5 mM MgCl₂, 1.0 mM EGTA, and 10 μg/ml of each leupeptin, aprotinin and pepstatin, and 1 mM phenylmethanesulfonylfluoride (PMSF)). All immunoblots were imaged using the Odyssey Imaging system and exported using Image Studio version 3.1.4 (LI-COR).

### Quantitative PCR

Calu-3 cells were seeded at a density of 3×105 cells/well in 12-well plates. Cells were infected with SARS-CoV-2 for an hour. Immediately after infection, mock-infected or infected cells were treated with DMSO or ASK1 inhibitor (GS-444217). Then, the cells were incubated for 24 hours for RNA extraction.

RNA extraction was performed using RNeasy Mini Kit (Qiagen) according to manufacturer's protocol. Two hundred nanograms of purified RNA was reverse transcribed using iScript gDNA Clear cDNA Synthesis Kit (Bio-Rad). To quantify SARS-CoV-2 genome levels, primers were designed to amplify a region (UpE) between ORF3a and E genes. Primer sequences used were SARS2 UpE F—ATTGTTGATGAGCCTGAAG and SARS2 UpE R—TTCGTACTCATCAGCTTG. All IL6, TNF-a and IL8 primers were purchased from Bio-Rad as indicated in the Materials and Methods section (Key Resources Table). Quantitative PCR reactions were performed with SsoFast EvaGreen supermix (Bio-Rad) assays.

### Cell Viability and Morphology Assay

Cell viability was measured using an MTT [3-(4,5-dimethyldiazol-2-yl)-2,5-diphenyl tetrazolium bromide] assay (Abcam, ab211091) as per manufacturer's protocol. Briefly, approximately 5000/well of THF cells were seeded into 96-well plates and incubated for 24 hr, and then cells were treated with either Selonsertib (50 μM) or GS444217 (50 μM) for 24 hr. Then cells were incubated with MTT reagent for 3 hours at 37° C. before adding MTT solvent and mixing on an orbital shaker for 15 mins. Absorbance was measured at OD590 nm and results were normalized to control culture conditions.

To monitor the cell morphology, Calu3 cells were infected with mock and I MOI of VSV for 1 hr. This was followed by treating the cells with DMSO or Selonsertib (50 μM). The cells were incubated for 24 hr, and their morphology was observed and imaged by EVOS microscope.

Autophagy and mTORC1 Experiments

THF or Calu3 cells were infected with mock or VSV-GFP for an hour, followed by adding DMSO, Selonsertib (50 μM), BIRB (10 μM), Torin1 (10 μM), MG132 (5 μM) or Chloroquine (20 μM) for 24 hr. The effect of Selonsertib on mTORC1 and autophagy was tracked by immunoblotting using antibodies against phospho S6K (phospho-p70) (T389) and LC3B respectively.

### Syrian Hamster In Vivo Study and Immunohistochemistry (IHC)

Four groups of Syrian Hamsters (n=3 male and 3 females in each group; total n=6/group) were used to investigate the physiological effect of ASK1 inhibition on SARS-CoV-2 infection. Infected groups of Syrian Hamsters were intranasal-challenged with SARS-CoV-2/SB3 at 10⁵ PFU. Selonsertib (100 mg/kg) or Vehicle (0.5% methylcellulose) were orally administrated for 7 days (2 days prior infection and 5 days after infection). Weight of hamsters was daily monitored for 7 days. At the end of day 7, all animals were sacrificed and lung tissues were collected and fixed in 10% neutral buffered formalin. Fixed tissues were sent to the McMaster Core Histology Facility for immunohistochemistry (IHC).

Slides were cut 4 um and were dewaxed and stained on Leica Bond Rx automated strainer. Then, they were treated with Epitope Retrieval 1 (Leica AR9961) for 20 minutes. Mouse anti-SARS/SARS-CoV-2 N (1:1000) were added to the slides in IHC super blocker (Leica PV6122) for 15 minutes. Secondary antibody (Rabbit anti-mouse) from Leica Bond Refine Polymer Kit (Leica DS9800) was preabsorbed with 20% normal hamster serum for an hour. Then, the slides were incubated with the ‘preabsorbed’ secondary antibody for 8 minutes. This was followed by tertiary antibody treatment using Goat anti-Rabbit polymer (Leica Bond Refine Polymer Kit, Leica DS9800) for 8 minutes. The slides were counterstained with Hematoxylin (Leica Bond Refine Polymer Kit), and they were dehydrated and coverslipped off line.

### Quantification and Statistical Analysis

Viral infection images of VSV-GFP, HSV-GFP, VacV-EYFP and HIV-GFP were scanned and quantified using Typhoon Imager. The IHC images were captured to HALO® software analysis (v3.5.3577.255). The slides were analyzed and quantified using HALO®'s Area Quantification module. For all quantified images, bars represent mean±s.e.m. p-values <0.05 were considered statistically significant and indicated in the respective Figure legends. *p<0.05; **p<0.01; ***p<0.001; ****p<0.0001)).

### Results

### ASK1/MKK3/MKK6/MKK4/p38MAPK Signaling Pathway is Activated by SARS-CoV-2 in Lung Cells, Calu3 Cells

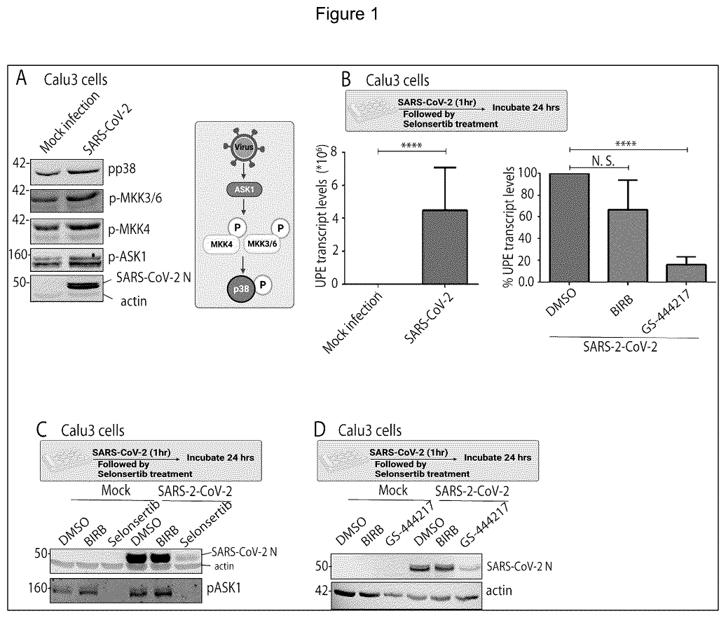

Various cellular stimuli including viral infections activate p38MAPK that play crucial roles in regulating cellular functions such as apoptosis and inflammation (Ono and Han 2000). Several studies showed that infections by several viruses can activate p38 MAP kinases (He et al., 2013, Law, Tam, Lee and Lau 2013, Leong, Ong and Chu 2015, Perfettini et al., 2005). Recently, the global phosphorylation profiles of a viral infection such as SARS-CoV-2 predicted the upregulation of the p38-MAPK signaling pathway (Bouhaddou et al., 2020). In accord, we measured the effect of SARS-CoV-2 infection on p38MAPK activation (FIG. 1A). The activation of ASK1/MKK3/MKK6/p38 signaling pathway was assessed by immunoblotting with antibodies. Our data showed that SARS-CoV-2 phosphorylates and activates p38MAPK and its upstream signaling ASK1/MKK3/MKK6/MKK4.

### ASK1 Inhibitors (Selonsertib and GS-444217) Block SARS-CoV-2 Replication and Subside the Storm of Several Cytokines Such as IL6, TNFα and IL8 in Lung Cells

### ASK1 Inhibitors Attenuate SARS-CoV-2 Replication in Calu3 Cells

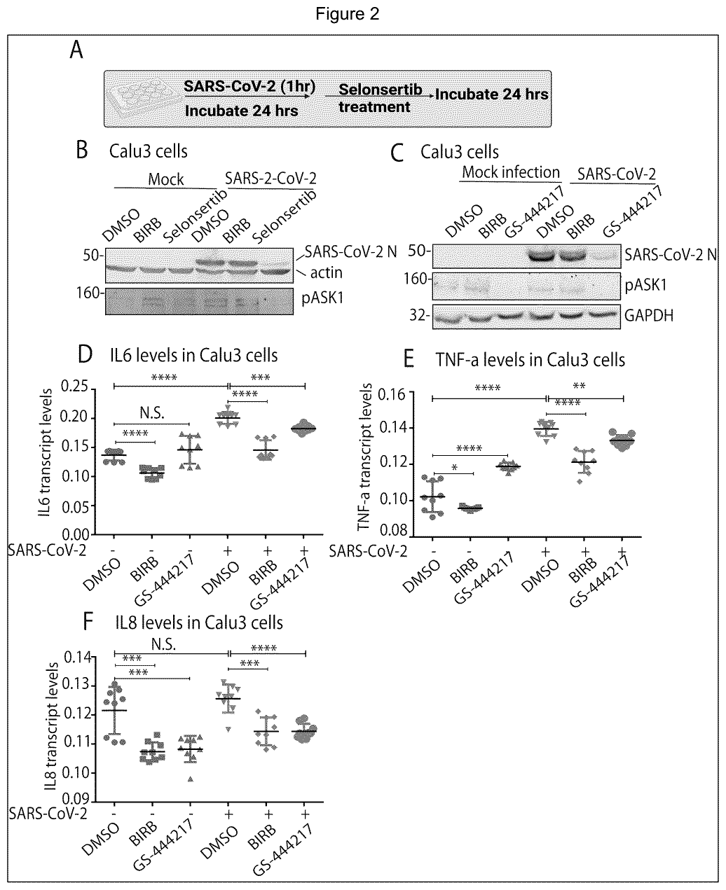

The effect of p38MAPK and ASK1 inhibition on viral replication were investigated. Thus, inhibiting p38 by Doramapimod (BIRB 796, for short BIRB) showed no antiviral properties against SARS-CoV-2 at 24 hpi. Intriguingly, inhibition of ASK1 by Selonsertib or GS-444217 drastically reduces SARS-CoV-2 replication at 24 hpi (FIG. 1 B-D and FIG. 2 A-C). The effect of ASK1 drugs was assessed by treating Calu3 cells with the drugs for 24 hours, and the drugs were added either after an hour (FIG. 1 B-D) or after 24 hours (FIG. 2 A-C) of SARS-CoV-2 infection. The inhibitory effect of ASK1 drugs on SARS-CoV-2 replication was determined by either measuring the SARS-CoV-2 genome level (FIG. 1B) by immunoblotting with antibodies against the SARS-CoV-2 nucleoprotein (FIGS. 1C and 1D, 2B and 2C). The data suggested that ASK1 inhibitors have antiviral properties against SARS-CoV-2, and highlighted the need of ASK1 for SARS-CoV-2 replication. To quantify SARS-CoV-2 genome levels, primers were designed to amplify a region (UpE) between ORF3a and E genes (see the resource table here or Banerjee, Rapin, Bollinger and Misra 2017).

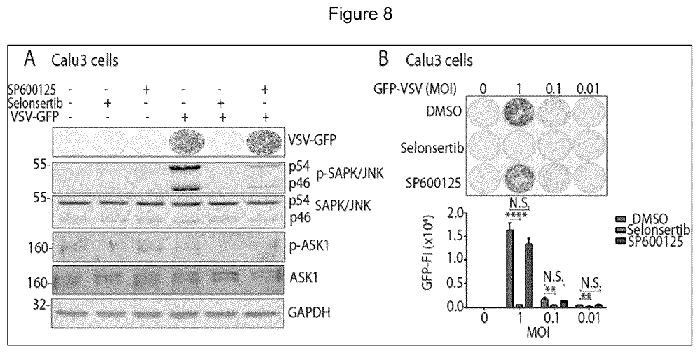

FIG. 8 shows the inhibition of ASK1 kinase decreases SARS-CoV-2 replication. (A) ASK1/p38MAPK is activated by SARS-CoV-2 infection. Calu3 cells were infected with SARS-CoV-2/SB3 for 24 h at MOI of 1. Then, the cells were lysed and collected at 24 hpi for immunoblotting. Schematic representation for ASK1/p38MAPK signaling pathway was depicted on the right side of the immunoblot. (B) ASK1 blockade (but not BIRB, the p38 MAPK inhibitor) reduces the levels of SARS-CoV-2 genome at 24 hpi. On the top panel, experimental outline that shows the treatment of ASK1 or p38MAPK inhibitors immediately after infection, then RNA was isolated to measure the SARS-CoV-2 genome levels by qPCR. (C and D) Immunoblots that show that blocking ASK1 by two inhibitors reduces the SARS-CoV-2 nucleoprotein. Calu3 cells were infected with SARS-CoV-2/SB3 for 24 h at MOI of 1. ASK1 inhibitors were added to the cells immediately after infection. Then, the cells were lysed and collected at 24 hpi for immunoblotting.

ASK1 Inhibitors Reduces Levels of Proinflammatory Cytokines Levels of IL6, TNFα and IL8 in Lung Cells Infected with SARS-CoV-2

Several studies showed SARS CoV-2 infection caused lung damage. Thus, severe cases of COVID-19 showed hyper-induction of proinflammatory cytokines storm including IL6, TNFα and IL8 (Banerjee et al., 2020, Olbei et al., 2021, Song et al., 2020). IL6−/− mice are characterized by less lung damage in viral lung infections (Birra et al., 2020). Hence, the effect of ASK1 and p38 drugs on IL6, TNFα and IL8 was measured. Indeed, Calu3 cells infected with SARS-CoV-2 show higher levels of IL6 and TNFα (but not IL8) (FIG. 2 D-F). As expected, both ASK1 (Selonsertib and GS-444217) and p38 (BIRB) drugs were able to diminish the increase levels of IL6, TNFα and IL8 cytokines in calu3 cells (FIG. 2 D-F). The data suggested the effect of ASK1 on these cytokines is downstream cascade via p38-MAPK. This also portrayed the dual role of ASK1 inhibitors that blocks SARS-CoV-2 replication and subsides a storm of proinflammatory cytokines.

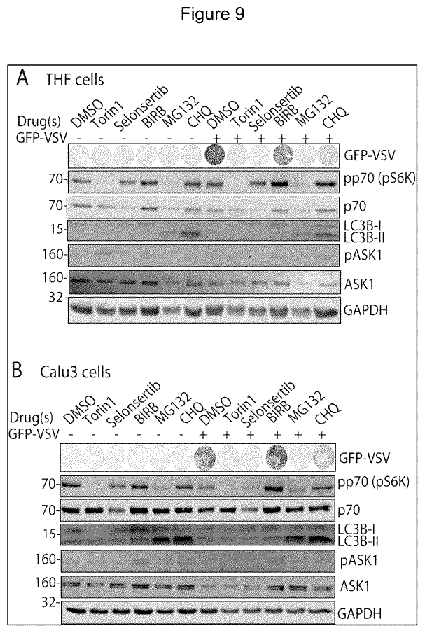

FIG. 9 shows ASK1 blockade reduces SARS-CoV-2 replication and the levels of proinflammatory cytokines, IL6, TNF-α and IL8. (A) The experimental outline for the treatment of ASK1 or p38MAPK inhibitors after SARS-CoV-2 infection. Briefly, cells were infected with SARS-CoV-2/SB3, ASK1 inhibitors were added to the infected cells after 24 h of infection. Then, the cells were either lysed and collected at 24 hpi for immunoblotting or RNA isolated to measure the cytokines levels by qPCR. (B,C) ASK1 blockade (but not BIRB, the p38 MAPK inhibitor) reduces the levels of SARS-CoV-2 genome at 24 hpi. (D, E, F) ASK1 and p38MAPK inhibitors reduces the transcript levels of IL6, TNF-α and IL8 in the infected cells.

### ASK1 Inhibition (by Selonsertib and GS-444217) Abrogates Replication of Several Viruses

The effect of ASK1 inhibitors was measured for several other viruses that use different mechanism for replication. Knowing that SARS-CoV-2 is a positive-stranded RNA virus, the drugs were tested on RNA viruses such as Vesicular stomatitis virus (VSV) that is a negative-sense RNA, which replicates in the cytoplasm or HIV that is a retrovirus, which carries single-stranded RNA into the DNA of host cell. The effect of ASK1 drugs was also assessed on DNA viruses such as Herpes Simplex Virus (HSV) that replicates in the nucleus or Vaccinia virus (VacV) that is DNA virus that replicates in the cytoplasm.

ASK1 Drugs Reduce the Replication of VSV, HSV and VacV

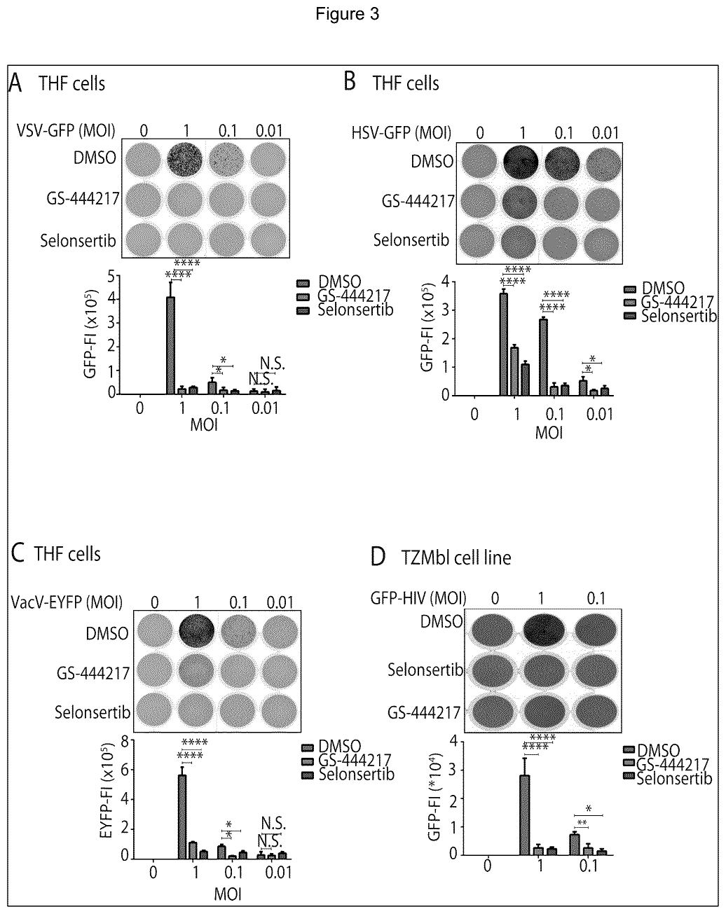

For testing effect of ASK1 drugs, Selonsertib and GS-444217 on VSV, HSV and VacV, two different cells were used; Calu3 (lung) and THF (fibroblast) cells. These cells were individually infected with either GFP-VSV, GFP-HSV or GFP-VacV at MOI of 1, 0.1 or 0.01. The GFP-fluorescent signals were captured and quantified by Typhoon Imager. FIG. 3 shows that Selonsertib and GS-444217 significantly abolish the GFP-intensity of VSV (FIG. 3A), HSV (FIG. 3B) and VacV (FIG. 3C) (the bars in the bar graph being in the order of the compounds shown (i.e. DMSO first, GS-444217 second and Selonsertib third). Indeed, these data highlight the role of host proteins such as ASK1 in viral replication, which are possibly hijacked to be a part of replication machinery for several viruses. This further suggests that ASK1 inhibitors are potential pan-antiviral drugs.

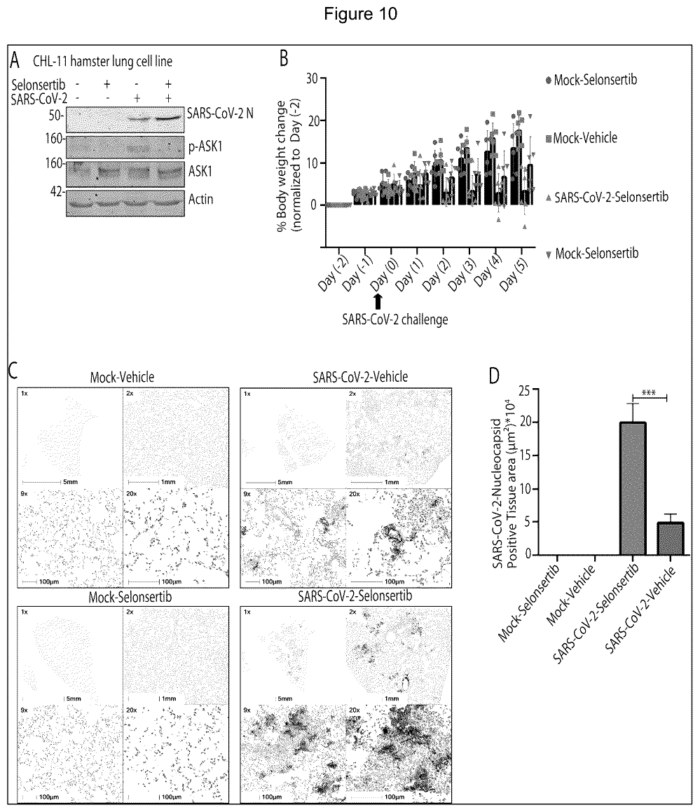

FIG. 10 shows ASK1 inhibition (by Selonsertib and GS-444217) abrogates replication of several viruses. (A-C) THF cells or (D) HeLa cells (TZMbl cell line) were infected individually with VSV-GFP, HSV-GFP, VacV-EYFP or HIV-1-GFP at MOI of 1, 0.1 or 0.01 followed by adding ASK1 drugs (GS-444217 or Selonsertib) at immediately after infection and cells were incubated for 24 h. Intensity of GFP signals was measured by Typhoon Imager.

### ASK1 Drugs Inhibit HIV Replication

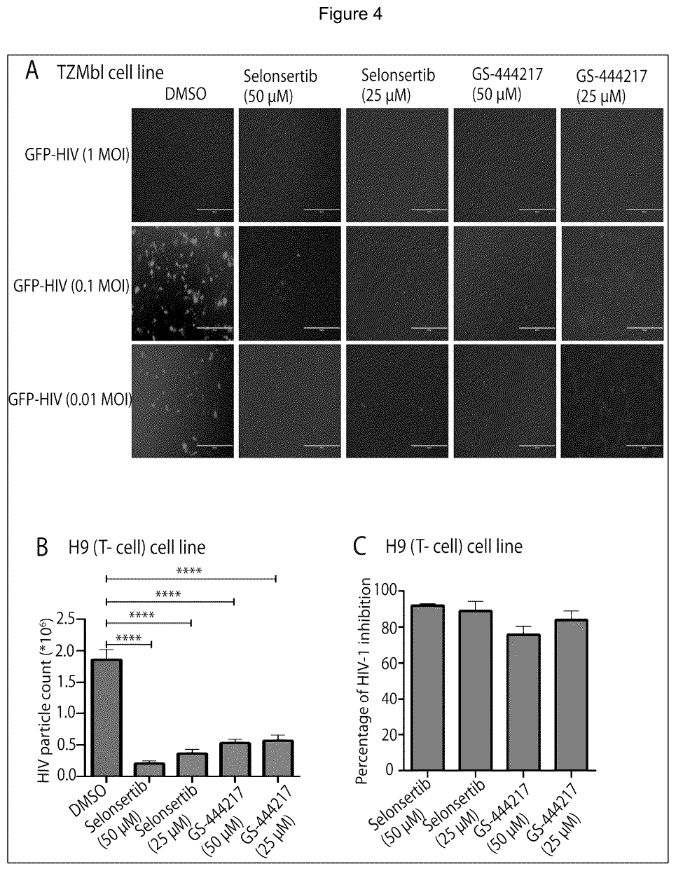

The human Immunodeficiency virus (HIV-1) is an RNA virus and very prone to mutations during its replication. These mutations help the virus to develop resistance against currently available therapeutics. Hunt for broad range antivirals is always required. The effect of ASK1 drugs to see their possible inhibitory effect on HIV-1 was investigated. Experiments were performed in two different ways firstly, mimicking primary infection in female genital tract and secondly looking at the effect of drugs on replication of HIV-1 in T-cells. To study effect of the two drugs (Selonsertib and GS-444217) on primary infection, TZMbl cell line was used, which is a human cervical cancer HeLa cell line, stably transfected with human CD4 receptor and CXCR4 and CCR5 coreceptors and expressing the β-galactosidase and firefly luciferase under control of the HIV-1 LTR promoter. We added the drugs TZMbl cells 2 hours and 24 hours after the HIV-1 infection and imaged cells under Typhoon to capture fluorescently labelled HIV-1 infected cells (FIG. 3D). The representative image of TZMbl cells is also shown in FIG. 4A.

To further test the effect of these drugs on HIV-1 replication, H9 cells were used which are chronically infected with HIV-1 III_{B}. The cells were either treated with DMSO or treated individually with the two drugs and supernatants were collected after 24 hours for viral titration. The results depicted that both drugs significantly reduced the viral replication in H9 cells (FIGS. 4B and 4C). Thus, ASK1 inhibitors are crucial for the replication of several viruses including HIV-1. This further support the potential role of ASK1 therapeutics as pan-antiviral drugs.

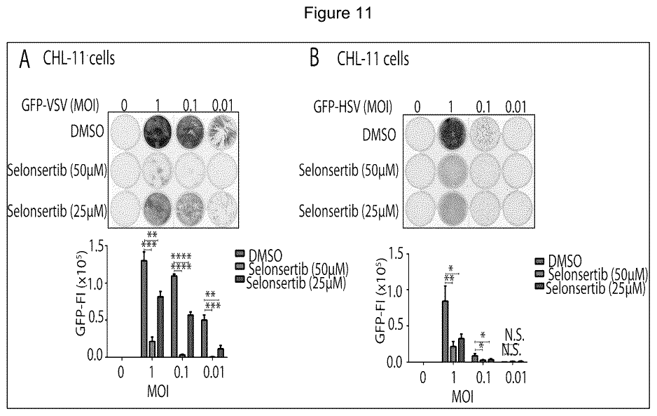

FIG. 11 shows ASK1 blockade inhibits HIV replication. (A) TZMbl cells were either mock infected or infected with fluorescence tagged HIV-1 NL4-3 GFP with MOI of 1 and 0.1 for 2 hours and then overlayed with medium containing Selonsertib or GS444217 in two different concentrations (50 or 25 μM). After 24 h of drug incubation, cells were imaged with EVOS fluorescence microscope. Representative images are shown. Magnification bar 400 μm. (B) viral supernatant collected from H9 cells were serially diluted in serum free medium and added to TZMbl cells, after 2 hours of incubation TZMbl cells were overlayed with serum containing medium and further incubated for 48 hours. After 48 hours of infection, media were aspirated, and the cells were fixed and stained for β-galactosidase activity. Blue infected cells showing positive β-galactosidase activity were counted and estimated accounting for dilution. (C) Percentage of HIV-1 inhibition was estimated from experiment (B).

ASK1/MKK3/6/MKK4/p38MAPK Signaling is a Key Regulatory Pathway that is Activated by Viral Infection

GFP-VSV was used as an example to infect cells in pretreament and posttreatment manners. Pretreament is described here as adding ASK1 inhibitors for a period of time (e.g. 24 h) and then, aspirating the drugs before viral infection. However, when the cells were treated with the drugs after infection, this would be termed as ‘posttreatment’ manner. Indeed, these experiments delineate the signaling pathway of ASK1/p38 that involved in viral infection (described below in more details), and proposed the lowest dose of ASK1 inhibitors that is required to inhibit the in vitro VSV replication.

FIG. 12 shows ASK1 drugs have a long-term effect that diminishes VSV replication and the downstream cascade signaling of p38MAPK. (A) Effect of ASK1 drugs on viral replication is dosage dependent. Calu3 cells were infected with VSV-GFP at MOI of 1, 0.1 or 0.01 followed by adding ASK1 (GS-444217 or Selonsertib) or p38MAPK drugs immediately after infection and cells were incubated for 24 h. Selonserib was added at different doses of a concentration of 10, 25 or 50 μM. (B,C) Calu3 or THF cells were pretreated with ASK1 inhibitors for 24 hours, then the drugs were aspirated and cleared before VSV infection, this followed by 24 hours of incubation. (A-C) Intensity of fluorescence (GFP-FI) or (EYFP-FI) signals was measured by Typhoon Imager. (D,E) Immunoblots show the acute and long-term effect of ASK1 blockade on the downstream cascade pathway of MKK3/MMK6/MKK4/p38MAPK. (D) Calu3 cells were treated in posttreatment (post) or pretreatment (pre) manners. In pretreament conditions, Calu3 cells were pretreated with ASK1 inhibitors for 24 hours, then the drugs were aspirated and cleared before VSV infection, this was followed by 24 hours of incubation. However, under posttreatment conditions, ASK1 inhibitors were added to the cells immediately after VSV infection, this was followed by 24 hours of virus incubation. (E) Calu3 cells were treated at different time points after VSV infection. For instance, at time point of 2 h, Selonsertib was added for 2 h immediately after infection. Then the drug containing media was aspirated and replaced with fresh media after 2 h. Representative of ASK1/p38MAPK signaling pathway was depicted on the right side of the immunoblots.

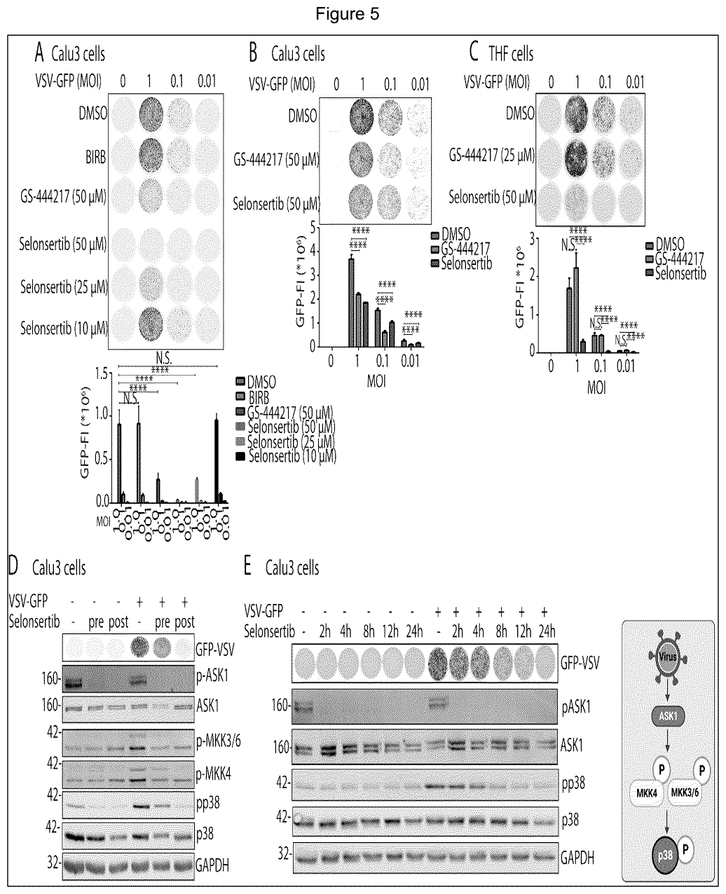

The experiments show the influence of different doses of ASK1 inhibitors, which is projected to be a dosage dependent effect. For instance, VSV replication is gradually decreased by adding increments of Selonsertib of a concentration above 10 μM in posttreatment manner (FIG. 5A). In contrast, the p38 inhibitor, BIRB has no obvious effect on VSV replication suggesting that ASK1 inhibitors might directly alter the viral replication. The work also demonstrates the long-term effect of ASK1 inhibitors that revealed when the cells were treated in pretreament manner at different doses (50 μM of Selonsertib or 50 or 25 μM of GS-444217), see FIG. 5B-D. The intriguing downstream effect of Selonsertib and GS-444217 on viral replication or on the phosphorylation of ASK1/p38MAPK lasts for 24 hours after aspirating drugs prior VSV infection (FIG. 5B-D). Nevertheless, the effect of ASK1 inhibitors is acute ever since VSV replication and p38MAPK phosphorylation start to weaken after 2 hours of adding Selonsertib to the infected cells (FIG. 5E). Taken together, this work reveals the ASK1/MKK3/6/MKK4/p38MAPK signaling is a key regulatory pathway in viral replication. In the bar graph of FIG. 5A, DMSO is the first bar, BIRB is the second bar, GS-444217 is the third bar, and Selonsertib at 50 μm, 25 μm and 10 μm are the fourth, fifth and sixth bars respectively).

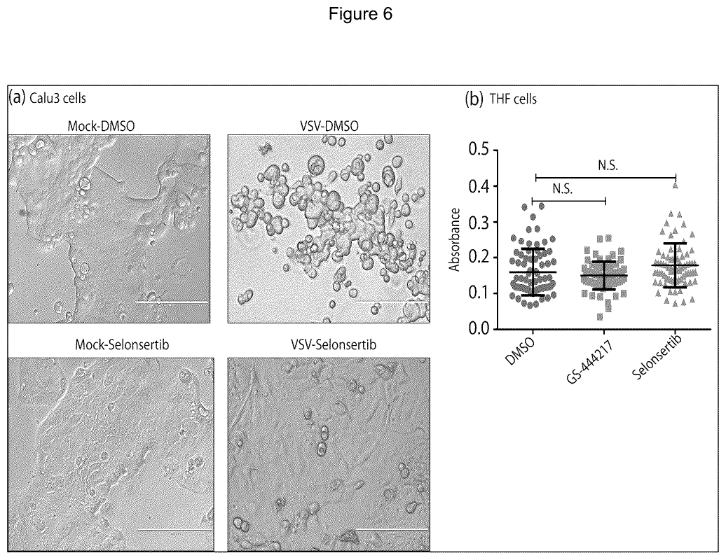

The effect of ASK1 inhibitors on cell viability was further evaluated by imaging and MTT assay. Blocking ASK1 activity by Selonsertib remarkably alleviates the changes in cell morphology caused by VSV replication (FIG. 6A). Indeed, the cells are healthy and safe in response to both GS-444217 and Selonsertib (FIGS. 6A and B).

FIG. 13 shows ASK1 Drugs (Selonsertib and GS-444217) do not alter the cell viability. (a) Calu3 cells was infected with 1 MOI of VSV in presence or absence of Selonsertib. (b) MTT assay shows that no difference between cells treated with DMSO, Selonsertib or GS-444217.

### ASK1 Knockout (KO) Reduces SARS-CoV-2 and VSV Replication

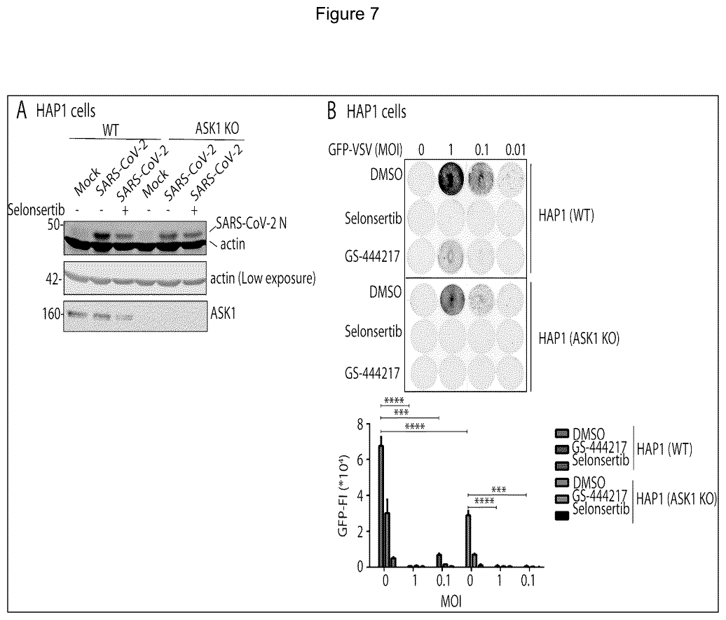

To study the physiological function of ASK1 on viral replication, ASK1 KO HAP1 cells (PerkinElmer) were used. HAP1 cells were infected with SARS-CoV-2 in presence or absence of Selonsertib. Immunoblot shows lower levels of the SARS-CoV-2 nucleoprotein in ASK1 KO cells in comparison to infected cells of HAP1 WT (FIG. 7A). There is also no significant difference between infected HAP1 KO cells treated or not with Selonsertib (FIG. 7A). The findings unveil ASK1 as important component for SARS-CoV-2 replication. Furthermore, the ability of VSV to replicate was lessened in ASK1 KO comparing the HAP1 WT. However, Selonsertib was still able to swipe the leftover of VSV infection in HAP1 KO cells, suggesting that other kinase might compensate the absence of ASK1, possibly ASK2, that is targeted by ASK1 inhibitors.

FIG. 14 SARS-CoV-2 and VSV replication is weakened in ASK1 depleted cells. (A) HAP1 cells (WT or ASK1 KO) were infected with SARS-CoV-2/SB3 for 24 h at MOI of 1 in presence or absence of Selonsertib. Then, the cells were lysed and collected at 24 hpi for immunoblotting. (B) HAP1 cells (WT or ASK1 KO) were infected with VSV-GFP at MOI of 1, 0.1 or 0.01 followed by adding ASK1 drugs (GS-444217 or Selonsertib) immediately after infection and cells were incubated for 24 h. In the bar graph, DMSO (first bar on left), GS-444217 (second bar), Selonsertib (right bar).

The Effect of Selonsertib is Independent on the Downstream Effectors of ASK1; p38MAPK and JNK

ASK1 is a key mediator of ROS-induced JNK and p38MAPK activation under pathological conditions (Kyriakis and Avruch, 2001). In accord, we measured the effect of p38MAPK and JNK inhibition on viral replication. First, the p38MAPK inhibitor, BIRB has no noticeable effect on VSV replication (FIG. 5A), suggesting that ASK1 inhibitor might alter the viral replication independently on p38MAPK. Likewise, JNK inhibition has no significant effect on VSV replication (FIGS. 8A and 8B). This indicates that ASK1 either directly affect VSV replication or via other pathways.

### Effect of Selonsertib on Protein Synthesis and Degradation

Several studies reported that the clinical use of Selonsertib is safe and tolerated in humans. Our data showed that Selonsertib relieved the drastic morphological changes that caused by viral infection (e.g. VSV), keeping the cell healthier (FIG. 6A). Moreover, Selonsertib did not alter the pathways that converge on protein synthesis (e.g. mTORC1) or degradation (e.g. autophagy) in Calu3 and THF cells (FIGS. 9A and 9B). Phosphorylation levels of p70 (S6K1), the downstream effector of the mammalian target of Rapamycin (mTORC1) is not changed in cells infected and treated with ASK1 in comparison to cells infected with VSV only (DMSO+VSV) (FIGS. 9A and 9B). The autophagy marker, LC3II is also not affected upon treating the infected cells with Selonsertib (VSV+Selonsertib). Together, these findings further support that ASK1 is hijacked by a virus to work as a part of its replication machinery in human.

### Selonsertib Enhanced In Vitro and In Vivo SARS-CoV-2 Replication in Lung Hamsters

The effect of Selonsertib on Syrian Hamster infected with SARS-CoV-2 was measured. First, the in vitro effect of ASK1 inhibition (by Selonsertib) on SARS-CoV-2 replication using CHL-11 hamster lung cell line was studied. In contrast to human data (FIGS. 1B-1D and 2A-2C), the level of SARS-CoV2 nucleocapsid was remarkably increased in the in vitro hamster lung cell line (FIG. 10A). This effect was further confirmed in the in vivo Syrian Hamster model (FIG. 10B-10D). The infected hamsters lost weight as expected, while control animals gained weight. Interestingly, the percentage of weight loss was ‘the most’ in the group of hamsters that was infected and treated with ASK1 inhibitor (SARS-CoV2-Selonsertib group) (FIG. 10B). Unlike human, blocking ASK1 augmented SARS-CoV-2 replication in hamster tissue. Further, IHC staining against SARS-CoV2 nucleocapsid in the hamster lung tissue showed the SARS-CoV2 dominated a spatial area of the lung tissue collected from SARS-CoV2-Selonsertib group (FIGS. 10C and 10D). Together, the in vitro and in vivo data suggest that Selonsertib have a differential effect on hamster in comparison to human.

Similar to human, Selonsertib has antiviral properties against VSV (FIG. 11A) and HSV (FIG. 11B) in hamster cell line. However, the intriguing effect of Selonsertib on SARS-CoV-2 in hamsters was opposite to that in human. Indeed, the in vivo and in vitro effect of Solensertib SARS-CoV2 that happened respectively in hamster lung tissue and lung cell line was similar (FIG. 10). This might suggest that the antiviral effect of Selonsertib is blocked by SARS-CoV-2 in hamsters.

While the present disclosure has been described with reference to what are presently considered to be the preferred examples, it is to be understood that the application is not limited to the examples described herein. To the contrary, the present disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### REFERENCES

Aguiar, J. A., Huff, R. D., Tse, W., Stämpfli, M. R., McConkey, B. J., Doxey, A. C., & Hirota, J. A. (2019). Transcriptomic and barrier responses of human airway epithelial cells exposed to cannabis smoke. Physiological reports, 7(20), e14249.
Banerjee, A., El-Sayes, N., Budylowski, P., Jacob, R. A., Richard, D., Maan, H., . . . & Mossman, K. (2021). Experimental and natural evidence of SARS-CoV-2-infection-induced activation of type I interferon responses. Iscience, 24(5), 102477.
Banerjee, A., Nasir, J. A., Budylowski, P., Yip, L., Aftanas, P., Christie, N., . . . & Mubareka, S. (2020). Isolation, sequence, infectivity, and replication kinetics of severe acute respiratory syndrome coronavirus 2. Emerging infectious diseases, 26(9), 2054.
Banerjee, A., Rapin, N., Bollinger, T., & Misra, V. (2017). Lack of inflammatory gene expression in bats: a unique role for a transcription repressor. Scientific reports, 7(1), 1-15.
Birra, D., Benucci, M., Landolfi, L., Merchionda, A., Loi, G., Amato, P., . . . & Moscato, P. (2020), COVID 19: a clue from innate immunity. Immunologic research, 68(3), 161-168.
Bouhaddou, M., Memon, D., Meyer, B., White, K. M., Rezelj, V. V., Marrero, M. C., . . . & Krogan, N. J. (2020). The global phosphorylation landscape of SARS-CoV-2 infection. Cell, 182(3), 685-712.
Bresnahan, W. A., Hultman, G. E, & Shenk, T. (2000). Replication of wild-type and mutant human cytomegalovirus in life-extended human diploid fibroblasts. Journal of virology, 74(22), 10816-10818.
Cuenda, A., & Rousseau, S. (2007). p38 MAP-kinases pathway regulation, function and role in human diseases. Biochimica et Biophysica Acta (BBA)-Molecular Cell Research, 1773(8), 1358-1375.
DeFilippis, V. R., Sali, T., Alvarado, D., White, L., Bresnahan, W., & Früh, K. J. (2010). Activation of the interferon response by human cytomegalovirus occurs via cytoplasmic double-stranded DNA but not glycoprotein B. Journal of virology, 84(17), 8913-8925.
Domínguez, J., del Mar Lorenzo, M., & Blasco, R. (1998). Green fluorescent protein expressed by a recombinant vaccinia virus permits early detection of infected cells by flow cytometry. Journal of immunological methods, 220(1-2), 115-121.
He, S. F., Wang, W., Ren, H., Zhao, L. J., & Qi, Z. T. (2013), Interferon alpha and ribavirin collaboratively regulate p38 mitogen-activated protein kinase signaling in hepatoma cells. Cytokine, 61(3), 801-807.
Ichijo, H., Nishida, E., Irie, K., Dijke, P. T., Saitoh, M., Moriguchi, T., . . . & Gotoh, Y. (1997). Induction of apoptosis by ASK1, a mammalian MAPKKK that activates SAPK/JNK and p38 signaling pathways. Science, 275(5296), 90-94.
Jindal, S., & Malkovsky, M. (1994). Stress responses to viral infection. Trends in Microbiology, 2(3), 89-91,
Kimpton, J., & Emerman, M. (1992). Detection of replication-competent and pseudotyped human immunodeficiency virus with a sensitive cell line on the basis of activation of an integrated beta-galactosidase gene. Journal of virology, 66(4), 2232-2239.
Kyriakis, J. M., & Avruch, J. (2001). Mammalian mitogen-activated protein kinase signal transduction pathways activated by stress and inflammation. Physiological reviews, 81(2), 807-869.
Law, A. H., Tam, A. H., Lee, D. C., & Lau, A. S. (2013). A role for protein phosphatase 2A in regulating p38 mitogen activated protein kinase activation and tumor necrosis factor-alpha expression during influenza virus infection, International journal of molecular sciences, 14(4), 7327-7340.
Leong, S. Y., Ong, B. K. T., & Chu, J. J. H. (2015). The role of Misshapen NCK-related kinase (MINK), a novel Ste20 family kinase, in the IRES-mediated protein translation of human enterovirus 71. PLoS pathogens, 11(3), e1004686.
Leveille, S., Goulet, M. L., Lichty, B. D., & Hiscott, J. (2011). Vesicular stomatitis virus oncolytic treatment interferes with tumor-associated dendritic cell functions and abrogates tumor antigen presentation. Journal of virology, 85(23), 12160-12169.
Minaker, R. L., Mossman, K. L., & Smiley, J. R. (2005). Functional inaccessibility of quiescent herpes simplex virus genomes. Virology journal, 2(1), 1-14.
Noyce, R. S., Taylor, K., Ciechonska, M., Collins, S. E., Duncan, R., & Mossman, K. L. (2011). Membrane perturbation elicits an IRF3-dependent, interferon-independent antiviral response. Journal of virology, 85(20), 10926-10931.
Olbei, M., Hautefort, I., Modos, D., Treveil, A., Poletti, M., Gul, L., . . . & Korcsmaros, T. (2021). SARS-CoV-2 causes a different cytokine response compared to other cytokine storm-causing respiratory viruses in severely ill patients. Frontiers in Immunology, 12, 381.
Ono, K., & Han, J. (2000). The p38 signal transduction pathway activation and function. Cellular signalling, 12(1), 1-13.
Perfettini, J. L., Castedo, M., Nardacci, R., Ciccosanti, F., Boya, P., Roumier, T., . . . & Kroemer, G. (2005). Essential role of p53 phosphorylation by p38 MAPK in apoptosis induction by the HIV-1 envelope. The Journal of experimental medicine, 201(2), 279-289.
Popovic, M., Sarngadharan, M. G., Read, E., & Gallo, R. C. (1984). Detection, isolation, and continuous production of cytopathic retroviruses (HTLV-III) from patients with AIDS and pre-AIDS. Science, 224(4648), 497-500.
Remy, G., Risco, A. M., Iñesta-Vaquera, F. A., González-Terán, B., Sabio, G., Davis, R. J., & Cuenda, A. (2010). Differential activation of p38MAPK isoforms by MKK6 and MKK3. Cellular signalling, 22(4), 660-667
Rozelle, D. K., Filone, C. M., Kedersha, N., & Connor, J. H. (2014). Activation of stress response pathways promotes formation of antiviral granules and restricts virus replication. Molecular and cellular biology, 34(11), 2003-2016.
Song, P., Li, W., Xie, J., Hou, Y., & You, C. (2020). Cytokine storm induced by SARS-CoV-2. Clinica chimica acta.
Wan, Q., Song, D., Li, H., & He, M. L. (2020). Stress proteins: the biological functions in virus infection, present and challenges for target-based antiviral drug development. Signal transduction and targeted therapy, 5(1), 1-40.
Wu, Y., Zhang, Z., Li, Y., & Li, Y. (2022). The Regulation of Integrated Stress Response Signaling Pathway on Viral Infection and Viral Antagonism. Frontiers in Microbiology, 12, 814635.

1. A method for the treatment or prevention of a viral infection in a subject in need thereof, comprising administering to the subject an effective amount of an apoptosis signal-regulating kinase 1 (ASK1) inhibitor.

2. The method of claim 1, wherein the ASK1 inhibitor is ASK1 inhibitor 10, selonsertib, GS444217, BPyO-34, GS-459679, GS-627, K811, K812, MSC2032964A, SRT-015, EP-027315, EP-026856, or Analog 21.

3. The method of claim 2, wherein the ASK1 inhibitor is selonsertib.

4. The method of claim 1, wherein the viral infection is an RNA virus infection or a DNA virus infection.

5. The method of claim 4, wherein the viral infection is a SARS-CoV-2 infection, a vesicular stomatitis virus infection (VSV), a herpes simplex virus infection, a human immunodeficiency virus infection or a vaccinia virus infection.

6. A method for the treatment or prevention of the symptoms, complications and/or disorders related to a cytokine storm in a subject in need thereof, comprising administering to the subject an effective amount of an apoptosis signal-regulating kinase 1 (ASK1) inhibitor.

7. The method of claim 6, wherein the ASK1 inhibitor is ASK1 inhibitor 10, selonsertib, GS444217, BPyO-34, GS-459679, GS-627, K811, K812, MSC2032964A, SRT-015, EP-027315, EP-026856, or Analog 21.

8. The method of claim 7, wherein the ASK1 inhibitor is selonsertib.

9. The method of claim 6, wherein the cytokine storm results from a viral infection, therapies, pathogens, cancer, smoking or autoimmune disorder.
